(19) United States
(12) Patent Application Publication
(10) Pub. No.: US 2006/0229636 A1
(43) Pub. Date: Oct. 12, 2006

(54) Expandable access device with mobility member

(76) Inventors: William N. Woodburn SR., Mantua, NJ (US); Brian R. Bankoski, West Grove, PA (US); Ralph C. Solitario JR., Douglassville, PA (US); Caryn E. Bundra, Downingtown, PA (US)

Correspondence Address: JONES DAY, 222 EAST 41ST STREET, NEW YORK, NY, 10017-6702, US

(21) Appl. No.: 11/368,131

(22) Filed: Mar. 2, 2006

Related U.S. Application Data
(60) Provisional application No. 60/659,050, filed on Mar. 4, 2005.

(51) Int. Cl. A61F 11/00

(52) U.S. Cl. 606/108

(57) ABSTRACT

An expandable access device for providing access to a surgical site and incorporating a mobility member. The expandable access device may have an access portion, an expandable portion, a mobility member connecting the access portion and the expandable portion, and a passageway extending therethrough. The mobility member may connect the access portion and the expandable portion so that the access portion may be translated and/or rotated relative to the expandable portion using a surgical or diagnostic instrument/device and/or a finger. The access portion may have a fixed cross-section or may be expandable. Moreover, the expandable portion of the expandable access device may have an unexpanded and expanded position. One or more tools may be inserted through the passageway and may be used to move the expandable portion between the expanded and unexpanded position. The expandable access device may be inserted into the body down towards a surgical site, with the expandable portion in the unexpanded position. Once in position, the expandable portion may be moved to the expanded position. A surgeon may insert instruments and/or devices through the passageway to the surgical site to perform a surgical procedure. Once the procedure has been completed, the expandable portion may be moved to the unexpanded position and removed from the body.

### CROSS REFERENCE TO RELATED APPLICATION

The present application claims priority to U.S. Provisional Application No. 60/659,050 filed Mar. 4, 2005, the entire content of which is expressly incorporated herein by reference thereto.

### FIELD OF THE INVENTION

The present invention relates to a device for providing access to a surgical site in the body and, more particularly, to an expandable access device which provides increased mobility.

### BACKGROUND OF THE INVENTION

It is known in the art to use cannulas and/or retractors to provide a surgeon with an access portal to a surgical site in a patient's body. These devices are especially useful in minimally invasive procedures, where an entire procedure is performed through one or more small incisions made in the patient. Minimally invasive procedures generally reduce the amount of trauma to the body and, thus, may reduce post-operative pain and recovery time. In particular, cannulas and/or retractors may be used to perform spinal operations, for example, discectomies, laminectomies, facectomies, pedicle screw fixation, etc.

Generally speaking, known cannulas include fixed diameter cannulas and expanding cannulas. A fixed diameter cannula may be in the form of a hollow tube having a diameter which remains constant from one end of the tube to the other. In use, a surgeon generally selects a cannula having a size which is large enough to perform part of or an entire surgical procedure. In this way, only a small incision is created through the patient's tissue. Fixed diameter cannulas provide surgeons with a limited range for moving surgical instruments and for visualizing the surgical site.

Expandable cannulas have an end which can expand proximate the surgical site, thereby stretching the tissue inside of the body. The other end of the expandable cannula generally protrudes from the body, thus providing surgeons with a fixed diameter access portal through which surgical instrument may be inserted into the body. An expandable cannula may provide a surgeon with more space to manipulate surgical instruments while, at the same time, allowing for only a small incision to be made through the tissue. Moreover, an expandable cannula may provide an increased field of vision to the surgical site.

Moreover, while retractors may provide surgeons with sufficient space to move surgical instruments as well as increased visibility of the surgical site, retractors generally cause more trauma to the body than expandable cannulas since retractors tend to stretch tissue from the skin down to the surgical site.

Thus, it is desirable to have an expandable device which provides a surgeon with the greatest ability to move surgical instruments and visualize a surgical site while, at the same time, causes the least amount of trauma to the body.

### SUMMARY OF THE INVENTION

The present invention relates to an expandable access device having an access portion, a expandable portion, a mobility member connecting the access portion and the expandable portion, and a passageway extending through the access portion and the expandable portion. The access portion may have a longitudinal axis, a proximal end, and a distal end. The proximal end of the access portion may be positioned outside of the body and a distal end which may be positioned inside the body. The access portion may provide a surgeon with a portal through which surgical or diagnostic instruments and/or devices may be inserted into the body. In one embodiment, the access portion may have a predetermined, fixed cross-sectional area. For example, the access portion may be a fixed dimension hollow tube which may have any shape, including circular, elliptical, square, rectangular, triangular or otherwise polygonal.

In some embodiments, the access portion may be expandable. For example, the access portion may be made of a flexible material (e.g., PEEK, polypropylene, sheet metal) such that the access portion may be deformed. The access portion may be temporarily deformed during the surgical procedure (e.g., by a surgeon's fingers or medical devices such as surgical or diagnostic instruments) or may be held in a deformed state using an instrument such as, for instance, a retractor. In another embodiment, the access portion may be a multi-piece assembly (i.e., formed of two or more pieces) which may be constructed of rigid material and which may be variably expanded and locked in any desired position. For example, the access portion may be moved and locked in increments between a collapsed/unexpanded position and a fully expanded position.

The expandable portion may be operably attached to the access portion. The expandable portion may have a longitudinal axis, a first (proximal) end and a second (distal) end. The first end of the expandable portion may be proximate the distal end of the access portion and the second end may be positionable proximate a surgical site. In one embodiment, the expandable portion may have an unexpanded position and an expanded position, wherein the second end of the expandable portion, in the expanded position, may have a larger cross-sectional area than in the unexpanded position. Surgical or diagnostic instruments and/or devices may be passed through the expandable portion, thereby enabling surgical or diagnostic instruments and/or devices to be inserted down to a surgical site.

The mobility member may attach the access portion and the expandable portion such that the access portion may have multiple degrees of freedom relative to the expandable portion. For example, the access portion may have six degrees of freedom of movement relative to the expandable portion—the access portion may translate along the x, y and/or z-axis, rotate about the z-axis (i.e., about the longitudinal axis of the access portion) and angulate about more than one axis in a plane which is transverse to the longitudinal axis of the access portion and/or the expandable portion (e.g., about the x and/or y-axis). The z-axis may be aligned with the longitudinal axis of the access portion and/or the expandable portion. An operator may perform any combination of translation, rotation and angulation independently and/or simultaneously. Using surgical or diagnostic instruments/devices and/or a finger, a surgeon may move the access portion in and out of the expandable portion and/or right, left, forwards or backwards relative to the expandable portion. Translation along the z-axis may allow a surgeon to manually palpate (i.e., examine by feel or by touch) patient anatomy during a surgical procedure. The mobility member may expand as the expandable portion moves to the expanded position and may contract as the expandable portion moves to the unexpanded position.

The mobility member may connect the access portion and expandable portion in any way so long as the access portion may move relative to the expandable portion. In one embodiment, the mobility member may be positioned around the access portion and the expandable portion. In particular, the mobility member may be positioned around a portion of the access portion and around a portion of the first end of the expandable portion. In another embodiment, the mobility member may extend towards the second end of the expandable portion such that the mobility member covers a majority of the expandable portion.

Upon expansion of the expandable portion, the mobility member may stretch to cover the expandable portion. Such a construction may be beneficial where the expandable portion is made of, for example, two or more pieces. As the pieces move apart, openings may be created between the pieces, thereby enabling tissue surrounding the expandable access device to enter into the passageway of the expandable access device. For example, in an embodiment where the expandable portion may have one or more blades, the blades may have edges such that in the unexpanded position, the edges of adjacent blades may be proximate each other and in the expanded position, the edges of adjacent blades may be a distance from each other, thereby creating openings between the blades. The mobility member may act as a barrier to prevent tissue (e.g., unresected tissue) from entering the passageway through the openings in the expandable portion.

In use, the expandable access device may be inserted into the body while the expandable portion may be in the unexpanded position. The expandable portion may be moved to the expanded position and one or more surgical or diagnostic instruments and/or device may be inserted into the passageway to perform a surgical procedure. A surgeon may then perform at least one of translating the access portion relative to the expandable portion, rotating the access portion relative to the expandable portion and angulating the access portion relative to the expandable portion. In one embodiment, a surgeon may perform at least one of rotating the longitudinal axes of the access portion and expandable portion relative to each other, translating the longitudinal axes of the access portion and expandable portion relative to each other and angulating the longitudinal axes of the access portion and expandable portion relative to each other. Angulation may be polyaxial angulation (e.g., angulation about any axis in the x and y plane). After the surgery is completed, the expandable portion may be moved to the unexpanded position and removed from the body.

### BRIEF DESCRIPTION OF THE DRAWINGS

The present invention can be better understood by reference to the following drawings, wherein like reference numerals represent like elements. The drawings are merely exemplary to illustrate certain features that may be used singularly or in combination with other features and the present invention should not be limited to the embodiments shown.

FIG. 1 is a perspective view of an exemplary embodiment of the device of the present invention in an expanded position;

FIG. 1A is a perspective view of an exemplary embodiment of an access portion of the present invention;

FIG. 2A is a perspective view of another exemplary embodiment of the device of the present invention;

FIG. 2B is a side view of the device of FIG. 2A in an expanded position;

FIG. 2C is a bottom view of the device of FIG. 2A in an expanded position;

FIG. 3A is a perspective view of an exemplary embodiment of an expandable portion of the device of FIG. 2A in an unexpanded position;

FIG. 3B is a rear view of the expandable portion shown in FIG. 3A in an unexpanded position;

FIG. 3C is a rear view of the expandable portion shown in FIG. 3A in an expanded position;

FIG. 4A is a perspective view of another exemplary embodiment of an expandable portion of the device of the present invention;

FIG. 4B is a perspective view of another exemplary embodiment of the device of the present invention in an expanded position;

FIG. 5 is a perspective view of another exemplary embodiment of the device of the present invention;

FIG. 6A is a perspective view of an exemplary embodiment of an expandable portion of the device of FIG. 5 in an expanded position;

FIG. 6B is a perspective view of an exemplary embodiment of a portion of the expandable portion of the device of FIG. 5;

FIG. 7 is a perspective view of an exemplary embodiment of the expandable portion of FIG. 6A in an unexpanded position;

FIG. 8A is a perspective view of another exemplary embodiment of the device of the present invention;

FIG. 8B is a perspective view of another exemplary embodiment of the device of FIG. 8A;

FIG. 9 is a perspective view of another exemplary embodiment of the device of the present invention in an unexpanded position;

FIG. 10 is a perspective view of the device of FIG. 9 in an expanded position;

FIG. 11 is a perspective view of the device of FIG. 9 in an expanded position with an alternative exemplary mobility member;

FIG. 12 is a perspective view of an exemplary embodiment of an expandable portion of the device of FIGS. 9-11 in an expanded position;

FIG. 13 is a perspective view of an exemplary embodiment of the expandable portion of FIG. 12 in an unexpanded position;

FIG. 14A is a perspective view of another exemplary embodiment of the device of the present invention; and

FIG. 14B is an alternative embodiment of the device of FIG. 14A.

### DETAILED DESCRIPTION

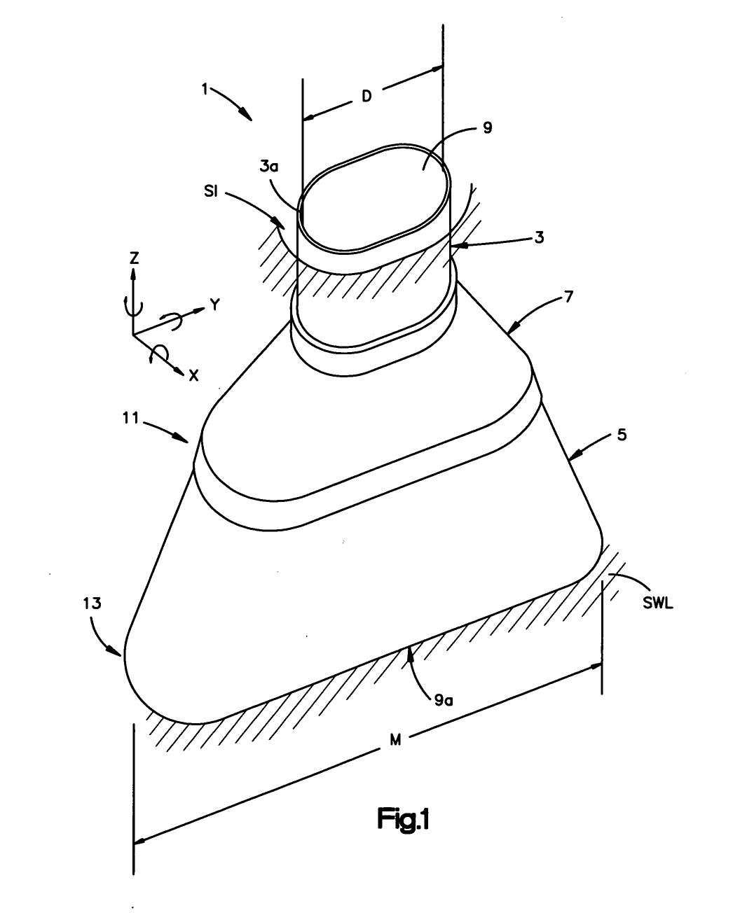

In general, as shown in FIG. 1, an expandable access device 1 of the present invention may have an access portion 3, an expandable portion 5, a mobility member 7 connecting the access portion 3 and the expandable portion 5, and a passage/passageway 9 extending through the access portion 3 and expandable portion 5 for receiving surgical or diagnostic instruments and/or devices. It should, however, be understood that those of ordinary skill in the art will recognize many modifications and substitutions which may be made to various elements of the present invention.

The expandable access device 1 may be used as a minimally invasive approach system for performing surgical procedures on the spine. For example, the expandable access device 1 may be used for anterior, lateral, anterio-lateral, and posterior approaches to the lumbar, thoracic and cervical areas of the spine. The expandable access device 1 may be used to perform, for example, pedicle screw fixation, discectomies, facectomies, laminectomies, etc. as well as a single level or multi-level posterior decompression/fusion procedure, including placement of pedicle screws, stabilization rods, and other devices. Moreover, the expandable access device 1 may also be used for non-fusion decompression and motion-preserving surgical procedures. Although the expandable access device 1 is described herein as being used in connection with spinal surgical procedures, one of ordinary skill in the art will readily appreciate that the device 1 may be used in any other part of the body to perform any surgical procedure or otherwise provide access from the exterior to the interior of a living body (e.g., for observation of anatomy). Thus, the location and/or surgical procedure is not intended to be limiting in any way. Furthermore, it will be appreciated by those skilled in the art that one or more expandable access devices 1 may be used to perform a procedure. For instance, in a bi-lateral procedure, a second expandable access device 1 may be used in parallel with a first expandable access device 1.

The access portion 3 and expandable portion 5 may be made of any bio-compatible material known in the art and is not intended to be limiting in any way. For example, the access portion 3 and expandable portion 5 may be made of steel, titanium, aluminum, an alloy, a composite, plastic, rubber, a polymer, etc. As will be readily appreciated by one of ordinary skill in the art, the access portion 3 and expandable portion 5 may be made of the same or different materials and may be radiolucent or radioopaque. In embodiments where the access portion 3 and/or expandable portion 5 may be radiolucent, radio-opaque markers (not shown) may be incorporated into or attached to the access portion 3 and/or expandable portion 5. The radio-opaque markers may assist a surgeon in properly aligning the access portion 3 and/or the expandable portion 5 relative to a patient's anatomy. With regard to the expandable portion 5, such a construction may provide the surgeon with the ability to visualize anatomical landmarks on an x-ray while, at the same time, providing the surgeon with a visual indication (i.e., the radio-opaque markers) of the position of the expandable portion 5 within the body. Various factors may be considered when determining the material used to make the access portion 3 and expandable portion 5, including but not limited to, for example, ability to withstand sterilization, weight, durability, resistance to staining, and the ability to grip the device 1, particularly with latex gloves.

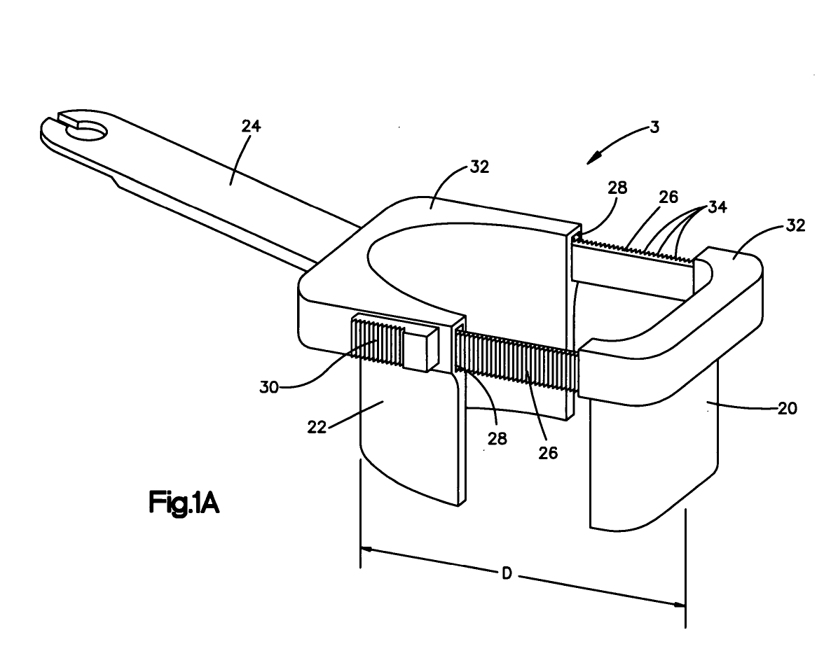

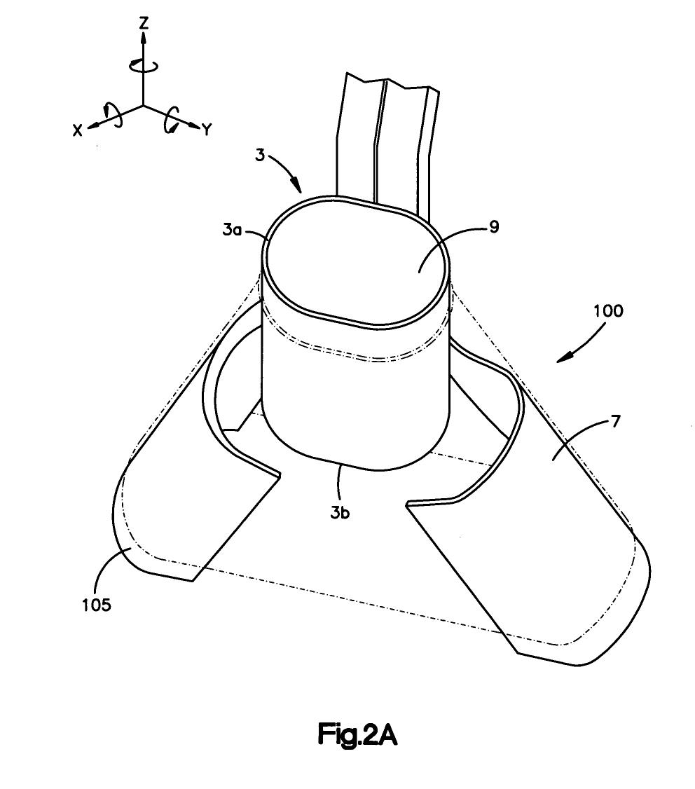

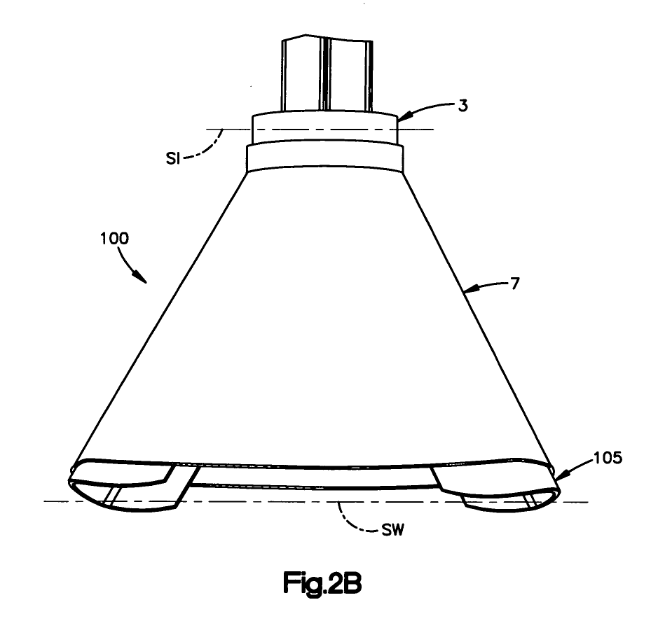

The access portion 3 may provide a surgical approach path through an incision formed in the skin. The access portion 3 may be a fixed dimension hollow tube (with a fixed cross-sectional area) having a proximal end 3a and a distal end 3b (FIG. 2A) wherein the proximal end 3a may be positioned outside of the body and above the skin incision SI while the distal end 3b may be positioned inside of the body and below the skin incision SI. The access tube 3 may be any shape known in the art including but not limited to, for example, circular, elliptical, square, rectangular, triangular, polygonal, etc. For example, the access tube 3 may be a substantially rigid tube (e.g., steel tube) having a wall thickness of between about 0.4 mm and about 0.6 mm and an outer dimension D of between about 10 mm and about 50 mm, preferably between about 20 mm and about 40 mm. As will be readily appreciated by one of ordinary skill in the art, the size of dimension D of the access portion 3 may be a factor of the surgical procedure being performed. It should be noted, however, that the access portion 3 may be expandable.

For example, the access portion 3 may be variably expandable along dimension D (i.e., the access portion 3 may have a variable dimension or variable cross-sectional area). In one embodiment, the access portion 3 may be made of a flexible material (e.g., PEEK, polypropylene, sheet metal) such that the access portion 3 may be deformed to elongate dimension D. The access portion 3 may be temporarily deformed during a surgical procedure (e.g., by a surgeon's fingers or a surgical or diagnostic instrument) or may be held in a deformed state using an instrument such as, for instance, a retractor.

In another embodiment, for example, as shown in FIG. 1A, the access portion 3 may be a multi-piece assembly, which may be constructed of rigid material and which may be variably expanded and locked in any desired position. For example, the access portion 3 may have a first portion 20 operably connected to a second portion 22, which may have a handle 24. In some embodiments, the access portion 3 may have no handle 24. It will be appreciated by those skilled in the art that the handle 24 may be used to attach the access portion 3 and, consequently, an expandable access device to an operating table. In particular, one end of a table mount (e.g., a rigid or flexible rod) (not shown) may be attached to the handle 24 and the other end of the table mount may be attached to an operating table. Moreover, surgical devices (e.g., a light, nerve root retractor) may be attached to an upper portion 32 of the access portion 3.

The first portion 20 of the access portion 3 may have one or more ratchet mechanisms 26 attached thereto. The ratchet mechanism 26 may be received in a receiving portion 28 of the second portion 22 so that the ratchet mechanism 26 may slide within the receiving portion 28. As shown in FIG. 1A, a locking mechanism 30 may be operably associated with the ratchet mechanism 26. The locking mechanism 30 may be moved between a locked position (e.g., away from the handle 24)—where the ratchet mechanism 26 and, consequently, the first portion 20 may be fixed with respect to the second portion 22—and an unlocked position (e.g., towards the handle 24)—where the ratchet mechanism 26 and, consequently, the first portion 20 may be moveable relative to the second portion 22.

The access portion 3 may be moved in increment between an expanded position and an collapsed/unexpanded position. In one embodiment, the access portion 3 may have a collapsed/unexpanded dimension D of about 30 mm which may be expanded in increments of about 2 mm to an expanded dimension D of about 44 mm. The access portion 3 may be locked in place at different increments so that a surgical procedure may be performed therethrough. In the embodiment of FIG. 1A, the increments may be a factor of the spacing between the teeth 34 of the ratchet mechanism 26.

An expandable access portion 3 may aid in visualization and/or access to a surgical site. In addition, an expandable access portion 3 may allow for use of surgical or diagnostic instruments (e.g., over-sized or odd sized surgical or diagnostic instruments) which otherwise may not have been able to be inserted and/or fit into a fixed diameter access portion 3. Furthermore, an expandable access portion 3 may enable a surgeon to perform more complex surgical procedures (e.g., 3-level fusion, 4-level fusion). An expandable access portion 3 may also accommodate a learning curve and the comfort level of surgeons performing a minimally invasive procedure. For example, a surgeon who may not be experienced with minimally invasive techniques may perform early procedures with the access portion 3 in an expanded position (e.g., expanding the access portion 3 so that the dimension D may be about 44 mm). In later procedures, as a surgeon becomes more experienced with minimally invasive techniques and/or as his/her skills improve, the surgeon may decrease the dimension D of the access portion 3 through which a procedure may be performed.

Referring again to FIG. 1, the expandable portion 5 may be operatively associated with the access portion 3 and may be any structure which may be moved between an unexpanded position and an expanded position such as, for example, those embodiments discussed below or, for example, as illustrated in U.S. Pat. No. 6,312,443. The expandable portion 5 may include a first end 11 and a second end 13 wherein the first end 11 may be positioned proximate the distal end 3b (FIG. 2A) of the access portion 3 and the second end 13 may be positioned distal the distal end 3b of the access portion 3 and adjacent to a surgical work level SWL. Moreover, the expandable portion 5 may have a cross-sectional area at the first end 11 and/or the second end 13 which may increase in size as the expandable portion 5 is moved from an unexpanded to an expanded position. In this way, the passage 9 through the expandable portion 5 may be increased in size to provide a larger working area 9a. Moreover, the expandable portion 5 may have a cross-sectional area at the first end 11 and/or the second end 13 which may decrease in size as the expandable portion 5 is moved from an expanded to an unexpanded position. Those skilled in the art will appreciate that one or more tools (not shown) may be used with an expandable portion to move the expandable portion between an expanded position and an unexpanded position.

Similar to the access portion 3, the expandable portion 5 may be any shape in the expanded and/or unexpanded position including, for example, circular, conical, elliptical, square, rectangular, triangular, polygonal, etc. In one preferred embodiment, the expandable portion 5, when in the unexpanded position, may have the same shape as the access portion 3. In the unexpanded position, the expandable portion 5 may have an outer dimension, for example, of between about 10 mm and about 50 mm, preferably between about 20 mm and about 40 mm. In the expanded position, the first end 11 of the expandable portion 5 may have a dimension, for example, of between about 10 mm and about 100 mm, preferably between about 40 mm and about 60 mm. Moreover, in the expanded position, the second end 13 of the expandable portion 5 may have a major dimension M, for example, of between about 30 mm and about 100 mm, preferably between about 80 mm and about 90 mm. It should be noted that the expandable portion 5 may be sized so that the expandable portion 5 may have a dimension larger than, equal to, or smaller than the dimension D of the access portion 3. As will be readily appreciated by one of ordinary skilled in the art, the size of the dimension at the first and/or second ends 11, 13 of the expandable portion 5 may be a factor of the surgical procedure being performed.

Moreover, the expandable portion 5 may be configured so that the expandable portion may be locked in place. In one embodiment, the expandable portion 5 may be opened to the fully expanded position and locked in place. In other embodiments, the expandable portion 5 may be variably expanded and locked in one or more intermediate positions between the collapsed/unexpanded position and the fully expanded position. For example, in an embodiment where the second end 13 of the expandable portion 5 has an unexpanded dimension M of about 30 mm and an expanded dimension M of about 85 mm, the expandable portion 5 may be locked in place at increments where the second end 13 may have a dimension of about 45 mm, 55 mm, 65 mm, 75 mm and/or 85 mm. It will be appreciated by those skilled in the art that any increment between locking positions may be used. For instance, the expandable portion 5 may be locked every 5 mm, 10 mm, 15 mm, 20 mm, etc. as the expandable portion 5 moves between the expanded and unexpanded position. In some embodiments, the expandable portion may move in varying increments (e.g., the second end 13 may be locked at a dimension M of 45 mm, 50 mm, 60 mm and 85 mm). A variably expanding expandable portion 5 may enable a surgeon to minimize retraction of tissue based on the surgical procedure being performed and the anatomy of a patient and, thereby, may minimize tissue trauma during a surgical procedure.

The access portion 3 and expandable portion 5 may be interconnected by a mobility member 7 which may loosely constrain the access portion 3 to the expandable portion 5 so that the access portion 3 may be able to move with respect to the expandable portion 5. In one embodiment, the mobility member 7 may be a thin sheet of elastomeric material. The mobility member 7 may be made of one or more components and may hold the access portion 3 in a predetermined position relative to the expandable portion 5 (i.e., a resting position). As best shown in FIG. 1, the mobility member 7 may be positioned around both the access portion 3 and the expandable portion 5. For example, the mobility member 7 may be positioned around a portion (e.g., the distal end 3b) of the access portion 3 and around the first end 11 of the expandable portion 5 extending down towards the second end 13 of the expandable portion 5. Alternatively, the mobility member 7 may be positioned within the access portion 3 and/or the expandable portion 5. For example, the mobility member 7 may be positioned around the access portion 3 and within the expandable portion 5 or around the expandable portion 5 and within the access portion 3. It should be noted, however, that the mobility member 7 may connect the access portion 3 and the expandable portion 5 in any way so long as the access portion 3 may move relative to the expandable portion 5. Moreover, the mobility member 7 may be connected to the access portion 3 and/or the expandable portion 5 by any method known in the art including, for example, adhesive, adhesive bonding, clips, pins, overmolding/overbonding, etc.

The mobility member 7 may have the ability to be stretched and/or manipulated by a surgical or diagnostic instrument, a surgical device, and/or a surgeon's finger(s) such that the access portion 3 may have multiple degrees of freedom with respect to the expandable portion 5. For example, the mobility member 7 may permit the access portion 3 to move in three-dimensions with respect to the expandable portion 5. That is, the mobility member 7 may allow the access portion 3 to move laterally with respect to the expandable portion 5, longitudinally with respect to the expandable portion 5, and in-and-out with respect to the expandable portion 5. More preferably, the mobility member 7 may permit the access portion 3 to have six degrees of freedom, including translation along the x, y and/or z-axis (e.g., up, down, left, right, front, back), rotation about the z-axis (i.e., about the longitudinal axis of the access portion 3) and/or angulation about more than one axis in a plane which is transverse to the longitudinal axis of the access portion 3 and/or the expandable portion 5 (e.g., about any axis in the x and/or y plane). An operator may be able to angulate the access portion 3 relative to the expandable portion 5 about more than one axis simultaneously (i.e., polyaxial angulation). Moreover, an operator may perform any combination of translation, rotation and angulation simultaneously.

The access portion 3 may translate from the resting position, for example, between about ±15 mm and about ±30 mm along the y and z-axes so that the access portion 3 may move a total distance of between about 30 mm and about 60 mm along the y and z-axes. The access portion 3 may translate from the resting position, for example, between about ±10 mm and about ±20 mm along the x-axis so that the access portion 3 may move a total distance of between about 20 mm and about 40 mm along the x-axis. Furthermore, the access portion 3 may rotate from the resting position, for example, between about ±30 degrees and about ±90 degrees about the z-axis so that the access portion 3 may rotate a total of between about 60 degrees and about 180 degrees about the z-axis. The access portion 3 may also rotate from the resting position, for example, between about ±45 degrees and about ±60 degrees about the x-axis so that the access portion 3 may rotate a total of between about 90 degrees and about 120 degrees about the x-axis. Moreover, the access portion 3 may rotate from the resting position, for example, between about ±20 degrees and about ±45 degrees about the y-axis so that the access portion 3 may rotate a total of between about 40 degrees and about 90 degrees about the y-axis. The access portion 3 may be independently and/or simultaneously rotated about, angulated about and translated along the x, y and/or z-axes. It will be appreciated, however, by those skilled in the art that that any combination of rotation, angulation and translation of the access portion 3 is envisioned.

Translation along the x, y and/or z-axis, rotation about the z-axis and/or angulation about the x and/or y-axis may enable a surgeon to manipulate surgical or diagnostic instruments and/or devices so as to allow, for example, for proper trajectory when inserting pedicles screws, fusion devices, etc. It should be noted that translation along the z-axis may also enable manual palpation of the anatomy during a surgical procedure (i.e., the surgeon may move the access portion 3 up and down with respect to the expandable portion 5 with his/her finger(s)).

The mobility member 7 may be sized and configured to prevent tissue intrusion into the passage 9. That is, the mobility member 7 preferably impedes soft tissue intrusion into at least a portion of working area 9a of the expandable portion. The mobility member 7 may be made from any material known in the art including, but not limited to, plastic, rubber, metal, a composite, etc. The mobility member 7 may be radiolucent or radioopaque and may be made of a material which may be transparent or opaque. Various factors may be considered when determining the material used to make the mobility member 7, including but not limited to the ability of the material to be stretched and return to its original size, ability to withstand sterilization, weight, durability, resistance to staining, and ability to grip the device 1, particularly with latex gloves which may be generally used during surgery. The mobility member 7 may be made from a flexible elastomeric material (natural or synthetic) or a polymer. For example, the mobility member 7 may be made of silicone rubber or urethane. Various factors may be considered when choosing a material for the mobility member including, for example, biocompatibility and the ability to allow translation and/or rotation of the access portion 3 relative to the expandable portion 5.

It should be noted that the access portion 3 and the mobility member 7 may be provided as a single, pre-assembly unit which may be sized and configured to engage any one of a number of different sized and configured expandable portions. The access portion 3 and the mobility member 7 may be manufactured as a disposable assembly, whereas the expandable portion 5 may be manufactured as a reusable assembly. In one embodiment, the entire expandable access device 1 may be manufactured as a disposable device. Alternatively, the entire expandable access device 1 may be manufactured as a reusable device, wherein each component may be capable of sterilization.

FIGS. 2A-2C illustrate an exemplary embodiment of an expandable access device 100. The expandable access device 100 may include an access portion 3, an expandable portion 105 and a mobility member 7. As previously described, the mobility member 7 may interconnect the access portion 3 to the expandable portion 105 so that the access portion 3 may be free to move with respect to the expandable portion 105. Furthermore, the mobility member 7 may be in the form of an elastic skirt so that the mobility member 7 may assist in maintaining the expandable portion 105 in a closed configuration.

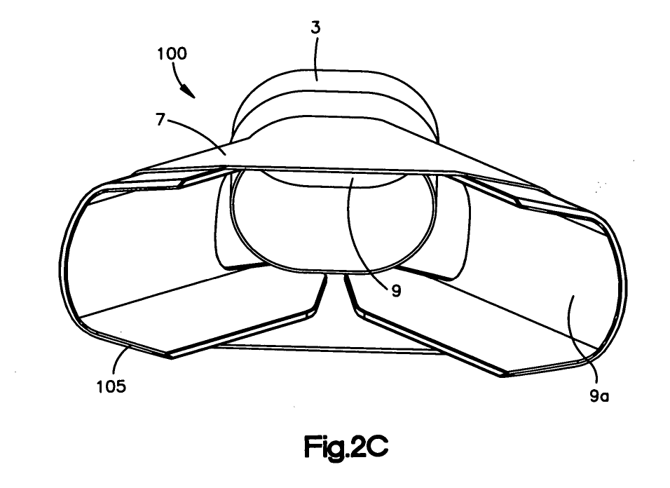

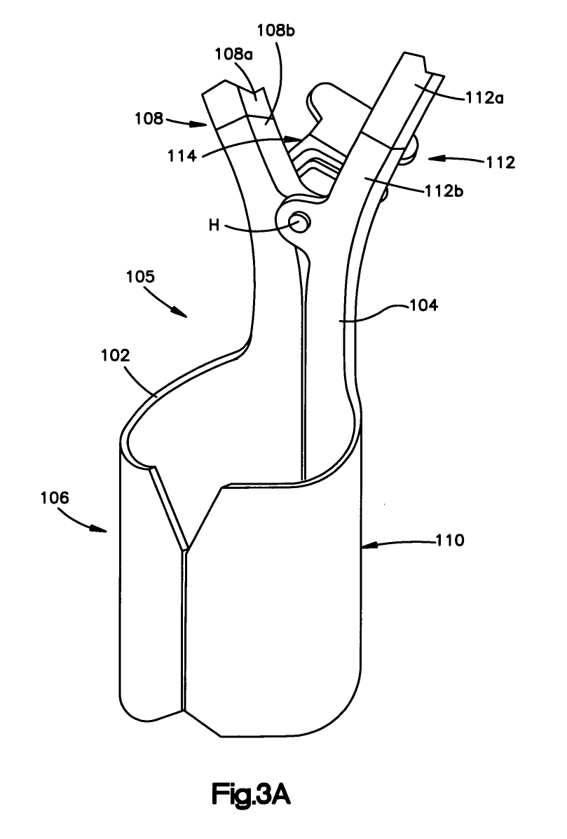

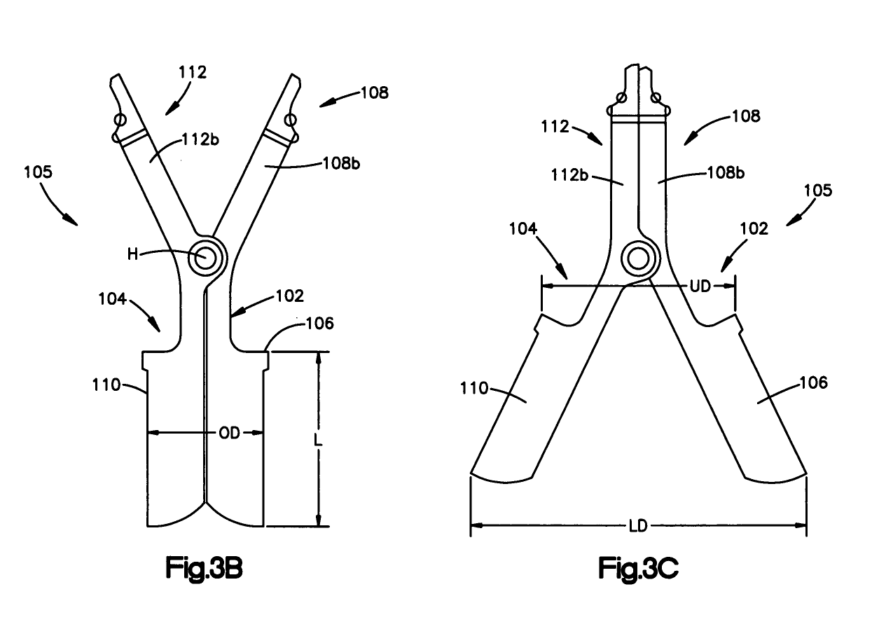

As shown in FIGS. 3A-3C, the expandable portion 105 may include a first member 102 and a second member 104 which may be pivotably connected at pivot point H. As shown, the first member 102 may have a first blade 106 and a first handle portion 108. Similarly, the second member 104 may have a second blade 110 and a second handle portion 112. The first and second blades 106, 110 may be curved (e.g., U-shaped, semi-circular) and may have a length L, for example, of between about 25 mm and about 70 mm, preferably between about 40 mm and about 50 mm. Those skilled in the art will appreciate that blades 106, 110 may be any shape (e.g. straight). Moreover, as will be readily appreciated by one of ordinary skill in the art, the length L may be a factor of the location in the body where the device 100 may be used to perform a surgical procedure. As best shown in FIGS. 3A and 3B, the expandable portion 105 may have, in the unexpanded position, an outer diameter OD, for example, between about 10 mm and about 50 mm, preferably between about 20 mm and about 40 mm.

In use, the expandable portion 105 of the expandable access device 100 may be inserted into a patient's body through an incision made in the patient's tissue. Preferably, the expandable portion 105 may be inserted into the patient in the unexpanded position. Thereafter, a surgeon may squeeze the handle portions 108, 112 thereby causing the blades 106, 110 to move away from each other so that the expandable portion 105 may be in the expanded position. The expandable portion 105 may also include a variable locking mechanism 114, which connects the first and second handle portions 108, 112 so that the blades 106, 110 may be moved with respect to one another and locked at selected increments.

As best shown in FIG. 3C, in the expanded position, the expandable portion 105 may have an upper dimension UD, for example, of between about 10 mm and about 100 mm, preferably between about 40 mm and about 60 mm. The expandable portion 105 may have a lower dimension LD, for example, of between about 30 mm and about 100 mm, preferably between about 80 mm and about 90 mm. Such a construction may provide access to the surgical work level SWL. As will be readily appreciated by one of ordinary skill in the art, the size of the upper dimension UD and/or lower dimension LD may be a factor of the surgical procedure being performed.

As best shown in FIG. 3A, the first and second handle portions 108, 112 may include upper handle portions 108a, 112a, respectively, which may be removable attached to lower handle portions 108b, 112b, respectively. It will be appreciated by one of ordinary skill in the art that the upper handle portions 108a, 112a may be connected to the lower handle portions 108b, 112b, respectively, in any manner known in the art including, but not limited to, for example, a bolt, a screw, a clip, etc. The lower handle portions 108b, 112b may include a pin extending from an end thereof for mating engaging a bore formed in the upper handle portions 108a, 112a. Alternatively, the upper handle portions 108a, 112a may incorporate a pin and the lower handle portions 108b, 112b may incorporate a bore. In use, the removable handles may enable the upper handle portions 108a, 112a to be removed from the lower handle portions 108b, 112b, respectively, once the expandable access device 100 has been inserted into the body and expanded. In this way, the upper handle portions 108a, 112a do not impede a surgeon's ability to perform a surgical procedure through the expandable access device 100. After a procedure is completed, the upper handle portions 108a, 112a may be re-attached to the lower handle portions 108b, 112b, respectively, so that the expandable portion 105 may be collapsed into the unexpanded position and removed from the body.

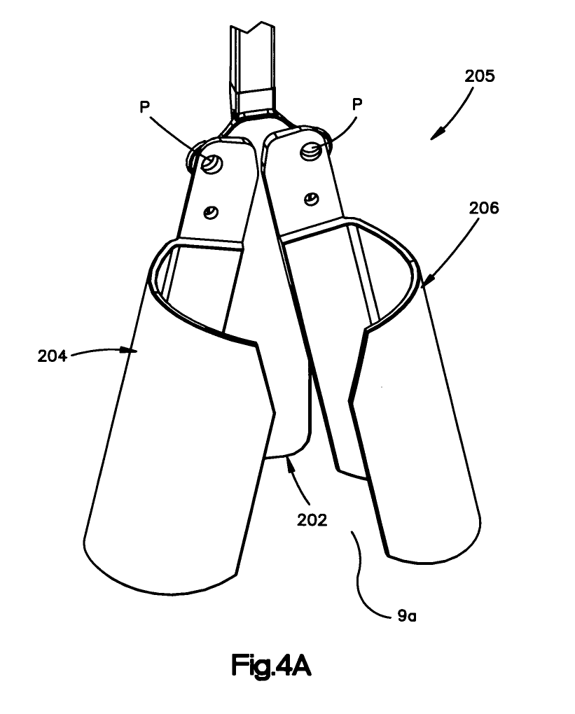

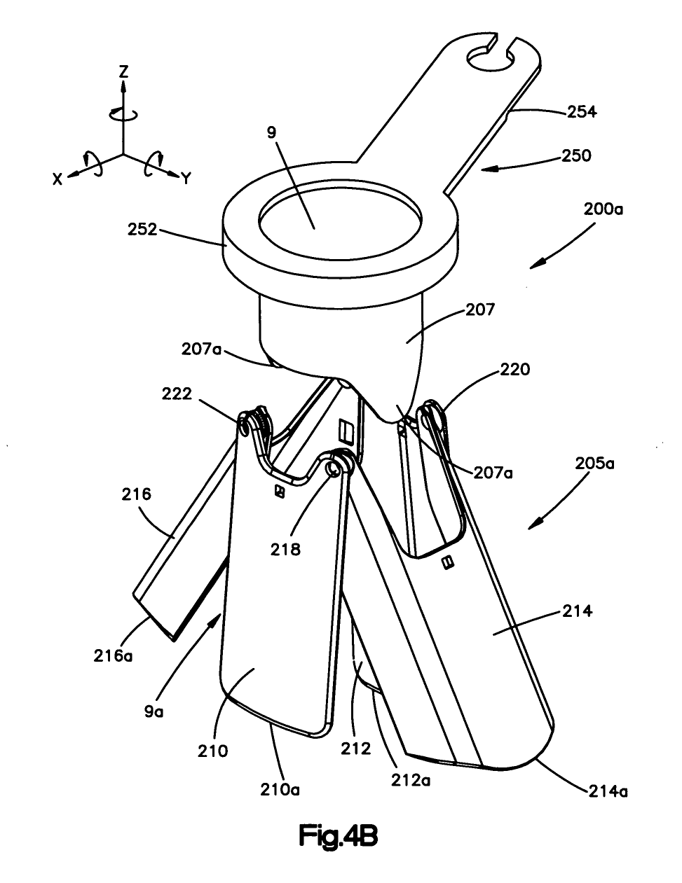

FIG. 4A illustrates another exemplary embodiment of an expandable portion—expandable portion 205. As shown, the expandable portion 205 may include a first blade 202, a second blade 204 and a third blade 206. The blades 202, 204, 206 may be curved (e.g., U-shaped, semi-circular) or straight. The blades 202, 204 and/or 206 may provide an enhanced barrier to soft tissue intrusion. The second and third blades 204, 206 may be pivotally connected to the first blade 202. As shown in FIG. 4A, the second and third blades 204, 206 may be pivotally connected to the first blade 202 at independent pivot points P, which may have axes which may be either parallel or at an angle (e.g., right angle, oblique) (i.e., the axes intersect each other) with respect to each other. In an embodiment where the axes of the pivot points P may be parallel to each other, the first blade 202 may remain stationary upon spreading the second and third blades 204, 206 away from each other. In an embodiment where the axes of the pivot points P may be at an angle with respect to each other, the first blade 202 may move away from the second and third blades 204, 206 as the second and third blades 204, 206 are spread apart. Alternatively, the second and third blades 204, 206 may be pivotally connected to the first blade 202 at a single pivot point. In use, one or more tools (not shown) may be inserted into the working area 9a and may be used to move the expandable portion 205 between the unexpanded position and the expanded position. As will be readily appreciated by one of ordinary skill in the art, the expandable portion 205 may include any number of blades.

For example, FIG. 4B illustrates an expandable access device 200a having four blades. As shown, the expandable access device 200a may include an access portion 207 and an expandable portion 205a. A mobility member (not shown) may interconnect the access portion 207 and the expandable portion 205a so that the access portion 207 may be free to rotate about, angulate about and/or translate along the x, y and/or z-axis. The access portion 207 may have extensions 207a which may provide an increased surface area for engaging the mobility member and which may assist in moving the mobility member as the access portion 207 is moved relative to the expandable portion 205a.

The expandable portion 205a may have a first blade 210, second blade 212, third blade 214 and fourth blade 216. The blades 210, 212, 214 and/or 216 may provide an enhanced barrier to soft tissue intrusion. The first blade 210 and the second blades 212 may be identically constructed (e.g., same size, shape, configuration). Moreover, the third blade 214 and the fourth blade 216 may be identically constructed. The third blade 214 and the fourth blade 216 may be pivotally connected to each of the first and second blades 210, 212. Specifically, the third blade 214 may be pivotally connected to the first blade 210 at pivot point 218 and to the second blade 212 at pivot point 220. Furthermore, the fourth blade 216 may be pivotally connected to the first blade 210 at pivot point 222 and to the second blade 212 at a pivot point (not shown). The pivot points 218 and 222 may have axes which may be either parallel or at an angle (e.g., right angle, oblique) (i.e., the axes intersect each other) with respect to each other. Similarly, the pivot point 220 and the other pivot point (not shown) of the second blade 212 may have axes which may be either parallel or at an angle (e.g., right angle, oblique) (i.e., the axes intersect each other) with respect to each other. In an embodiment where the axes of the pivot points may be parallel to each other, the first blade 210 and/or second blade 212 may remain stationary upon spreading the third and fourth blades 214, 216 away from each other. In an embodiment where the axes of the pivot points may be at an angle with respect to each other, the first blade 210 and the second blade 212 may move away from each other as the third and fourth blades 214, 216 are spread apart. In such a construction, the distal ends 210a, 212a of the first and second blades 210, 212, respectively, may move apart from each other, and the distal ends 214a, 216a of the third and fourth blades 214, 216, respectively, may move apart from each other.

When the first and second blades 210, 212 and/or the third and fourth blades 214, 216 may be moved apart, the size of the working area 9a may be enlarged. In use, one or more tools (not shown) may be inserted through the passage 9 and into the working area 9a and may be used to move the expandable portion 205a between the unexpanded position and the expanded position.

As shown in FIG. 4B, a handle member 250 may be attached to the access portion 207. The handle member 250 may be a separate piece or integrally formed with the access portion 207 (the handle member 250 and the access portion 207 may be a single piece, for example, as shown in FIG. 1A). Moreover, the handle member 250 may be used to connect the access portion 207 to an operating table so that the expandable access device 200a may be held in place relative to the patient's body during surgery. The handle member 250 may include a ring 252 and a flange 254 extending from the ring 252. The ring 252 may be connected to/positioned around the access portion 207 and a table mount (not shown) (e.g., a rigid or flexible rod) may be attached to the flange 254 to fix the handle member 250 to an operating table. It will be appreciated by those skilled in the art that the handle member 250 may be used with any embodiment of an expandable access device to connect the expandable access device to an operating table. Additionally, the handle member 250 may be sized and configured so that surgical or diagnostic instruments (e.g., a light, nerve root retractor) may attached thereto.

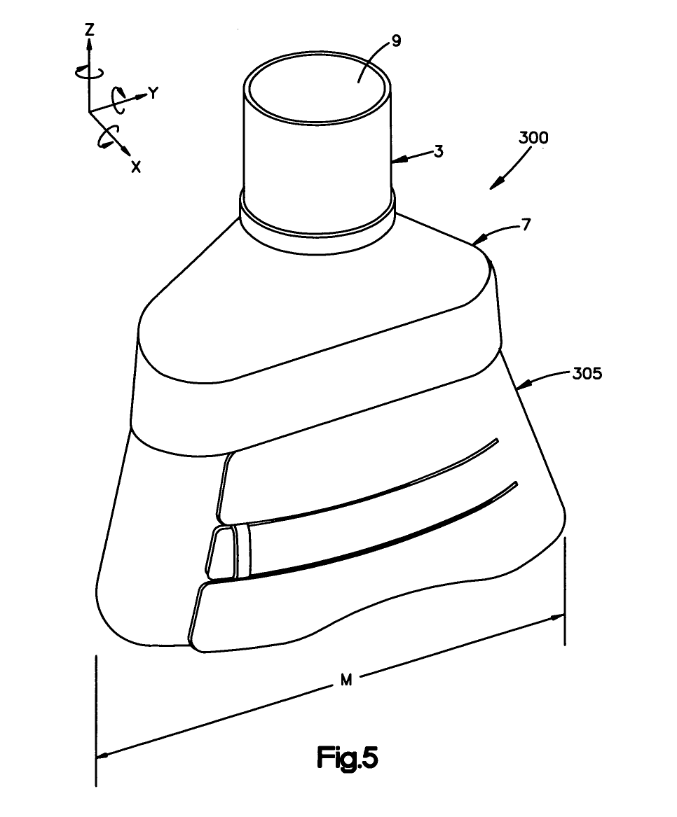

FIG. 5 shows an alternative embodiment of an expandable access device—expandable access device 300. As shown, the expandable access device 300 may include an access portion 3, an expandable portion 305, and a mobility member 7, which as previously described, may connect the access portion 3 to the expandable portion 305 so that the access portion 3 may be free to move with respect to the expandable portion 305. The device 300 may further includes a passage 9 extending therethrough.

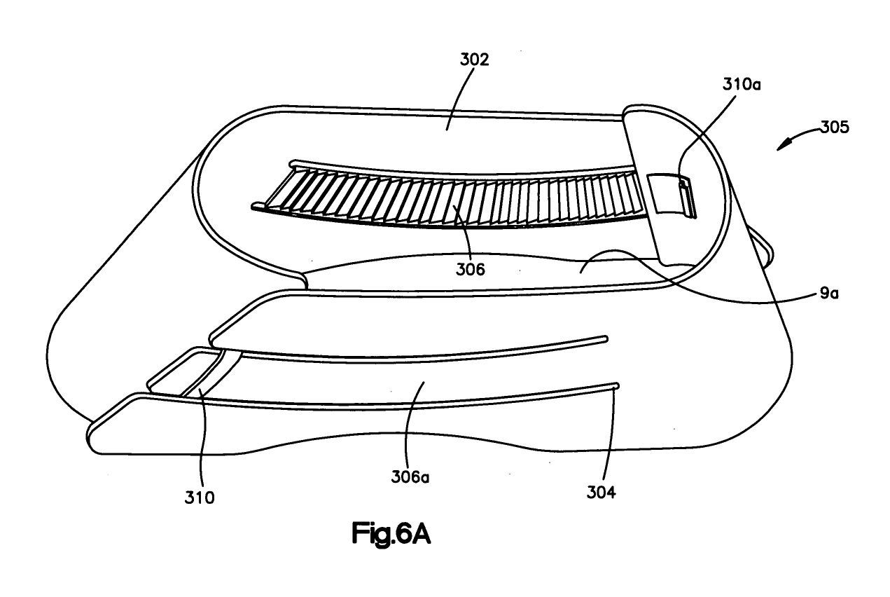

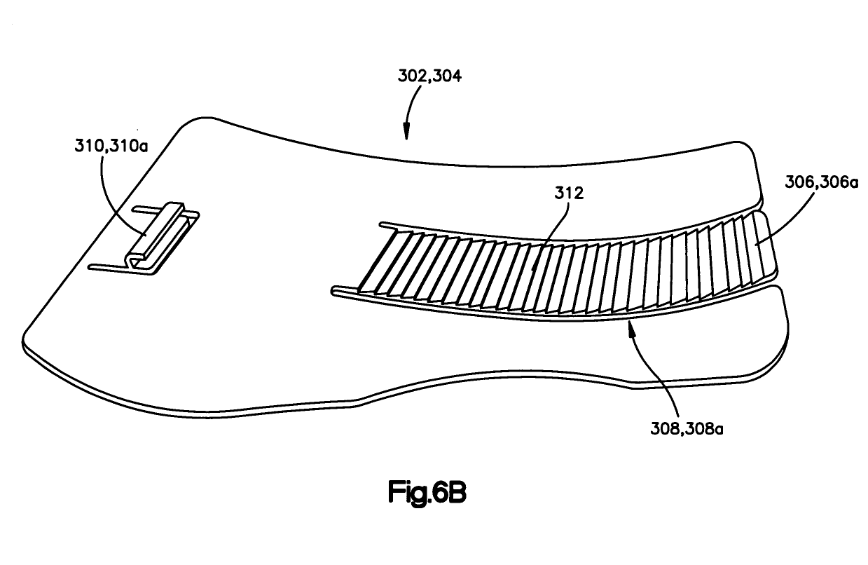

As shown in FIG. 6A, the expandable portion 305 may include first and second members 302, 304, which may be identically constructed. Preferably, the first and second members 302, 304 may be flexible, for example, the first and second members 302, 304 may be made from, for example, a flexible biocompatible polymer (e.g., PEEK, polypropylene) or a thin piece of sheet metal so that the first and second members 302, 304 may be contourable. As best shown in FIGS. 6A and 6B, the first and second members 302, 304 may each include a receiving portion 310, 310a, and a flap 306, 306a, which may define a deployment path 308, 308a. The first and second members 302, 304 may be sized and configured so that the flap 306 of the first member 302 may be inserted into the receiving portion 310a of the second member 304. Similarly, the flap 306a of the second member 304 may be inserted into the receiving portion 310 of the first member 302. The flap 306, 306a may include a plurality of teeth 312 formed thereon to matingly engage the receiving portion 310a, 310, respectively, so that, in use, the receiving portions 310, 310a may be moved along the deployment path 308a, 308, respectively, and may be selectively fixed along a length thereof. The deployment paths 308, 308a may be curved and may provide for optimal deployment kinematics. Such a construction may enable the first and second members 302, 304 to translate (slide) and rotate relative to each other as the expandable portion 305 moves between the expanded and unexpanded position. In use, one or more tools may be inserted into the passage 9 to expand and/or collapse the expandable portion 305.

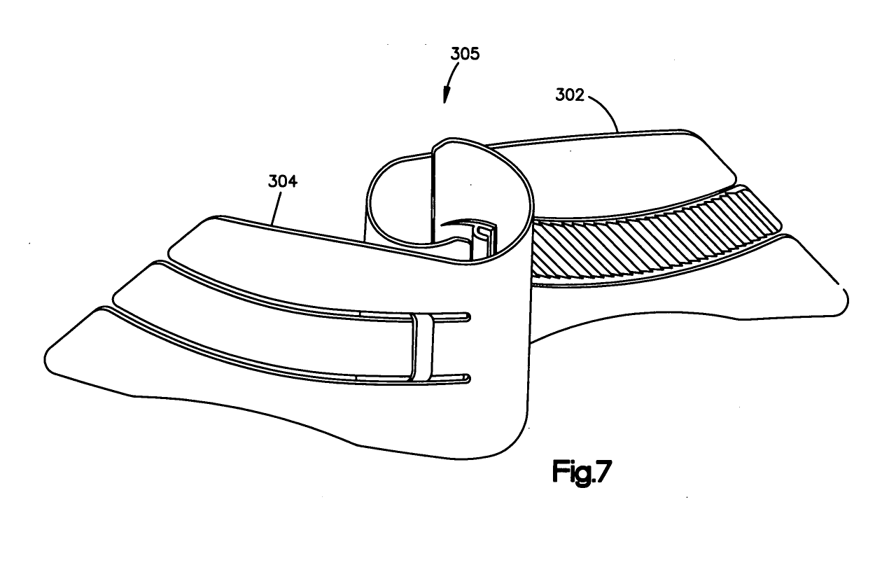

FIG. 7 illustrates the expandable portion 305 in an unexpanded state with the flexible sides of the first and second members 302, 304 extending away from a central tubular geometry. The flexible sides of the first and second members 302, 304 may be wrapped around themselves and may be retained in a substantially tubular geometry by the mobility member 7. The expandable portion 305 may collapse to an unexpanded position so that the outer dimension of the expandable portion 305 may be similar to the outer dimension D of the access portion 3. In the expanded position, as best shown in FIGS. 5 and 6A, the expandable portion 305 may expand and be in the form of an oval. In its expanded position, the expandable portion 305 may have a major dimension M, which may be at least about 85 mm. As will be readily appreciated by one of ordinary skill in the art, the size of dimension M may be primary based upon the location and the surgical procedure being performed.

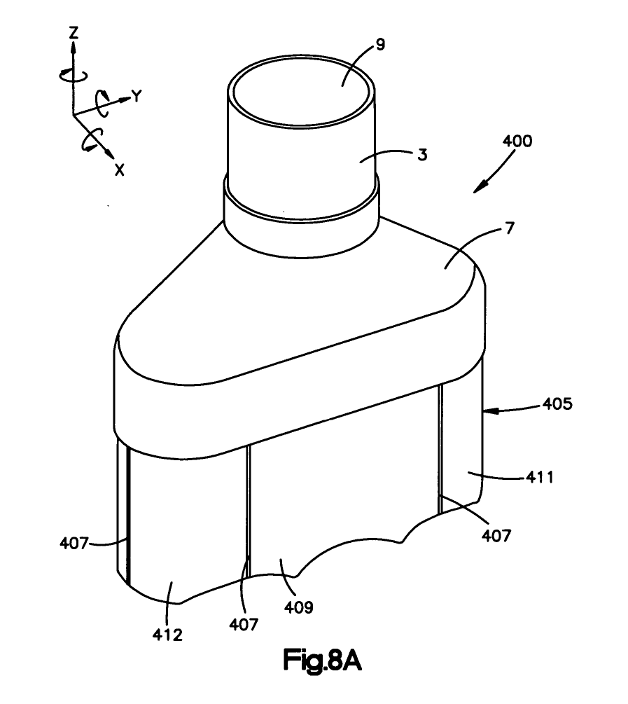

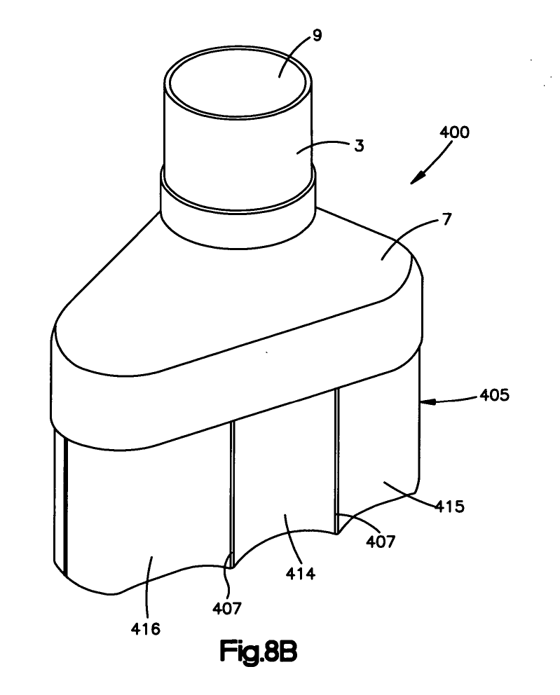

FIG. 8A illustrates yet another embodiment of an expandable access device of the present invention. As shown, the expandable access device 400 may include an access portion 3, an expandable portion 405, a mobility member 7 which may connect the access portion 3 and the expandable portion 405 so that the access portion 3 may be free to move with respect to the expandable portion 405. The expandable access device 400 may also include a passage 9 extending therethrough.

As will be readily appreciated by one of ordinary skill in the art, the expandable portion 405 may be any structure which may collapse to an unexpanded position by folding upon itself (e.g., so that the expandable portion 405 may be flat, cylindrical, etc.). For example, in one embodiment of the device 400, the expandable portion 405 may have one or more “living hinges.” In such a construction, the expandable portion 405 may be made of a single piece of bio-compatible material into which one or more hinges 407 may be formed, molded or cut. The expandable portion 405 may be made of any material known in the art including, for example, a biocompatible, flexible polymer (e.g., PEEK, polypropylene). The hinges 407 may be thinned portions formed in the material and, thus, may be flexible. The hinges 407 may also act as springs, which may allow the expandable portion 405 to flex as well as bias the expandable portion 405 towards an expanded position. The “living hinge” arrangement may also eliminate the need to have two pieces of material joined together by a separate hinge piece. It should be noted, however, that other embodiments of the expandable portion 405 may have one or more separate hinges which may join two or more pieces of material. Moreover, the “living hinge” arrangement may enable the expandable portion 405 to expand and contract without creating openings, spaces or holes in the expandable portion 405.

The expandable portion 405 may have a first wall portion 409, a second wall portion 411 and a third wall portion 412. An identical set of wall portions (not shown) may form the remainder of the expandable portion 450 and may be connected by hinges 407 to the second and third wall portions 411, 412. Hinges 407 may be disposed between the first wall portion 409 and second and third wall portions 411, 412. Those skilled in the art will appreciate that hinges 407 may be positioned in different locations on the expandable portion 405. For example, as shown in FIG. 8B, the hinges 407 may be positioned so that a first wall portion 414 may be positioned between second and third wall portions 415 and 416.

In use, the expandable portion 405 may be configured to fit within a tube (e.g., a cannula) (not shown) so that the expandable access device 400 may be inserted into the body. The expandable portion 405 may fold onto itself so that the expandable portion 405 may have an outer dimension or diameter, for example, of at least about 30 mm. In order to insert the expandable access device 400 in the body, the expandable portion 405 and/or the access portion 3 may be positioned within a tube such that the expandable portion 405 may be in an unexpanded position. It will be appreciated, however, that any other structure which may hold the expandable portion 405 in the unexpanded position may be used. When the tube and expandable portion 405 are positioned proximate a surgical site in the body, the tube may be withdrawn from the body and, thereby, may allow the expandable portion 405 to move to the expanded position. A surgical procedure may be performed through the expanded device.

In order to remove the expandable portion 405 from the body, the tube may be re-inserted into the body over the access portion 3 and the expandable portion 405. As the tube moves over the expandable portion 405, the expandable portion 405 may move to an unexpanded position. Once the expandable portion 405 is moved to an unexpanded position and/or contained within the tube, the tube and the expandable access device 400 may be removed from the body. In another embodiment, the expandable portion 405 may be flattened for insertion into and/or removal from the body. It will be appreciated by those skilled in the art that one or more instruments may be used to move the expandable portion 405 to the expanded and/or unexpanded position.

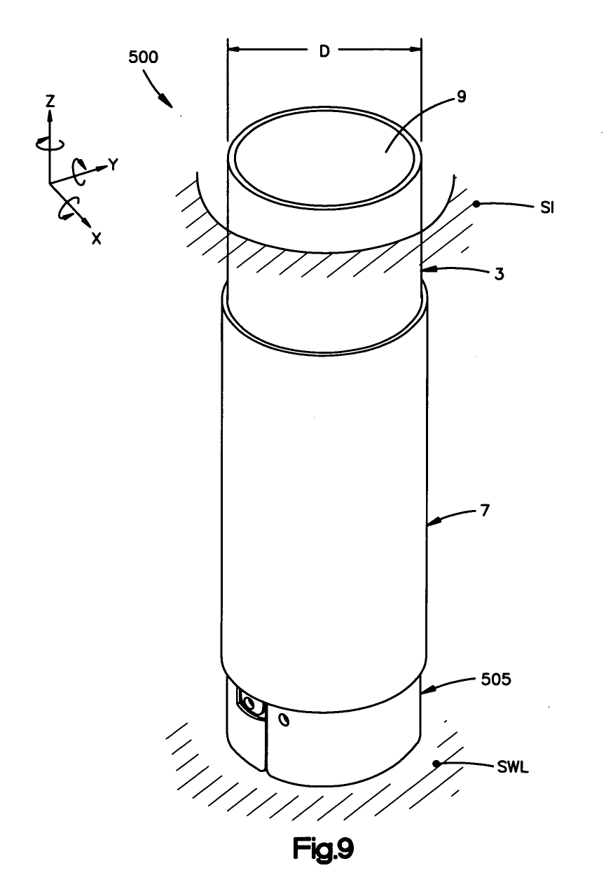

FIGS. 9-13 illustrate another embodiment of an expandable access device—expandable access device 500. The expandable access device 500 may have an access portion 3, an expandable portion 505, and a mobility member 7, which may connect the access portion 3 and the expandable portion 505 so that the access portion 3 may be free to move with respect to the expandable portion 505. The expandable access device 500 may also include a passage 9 extending therethrough.

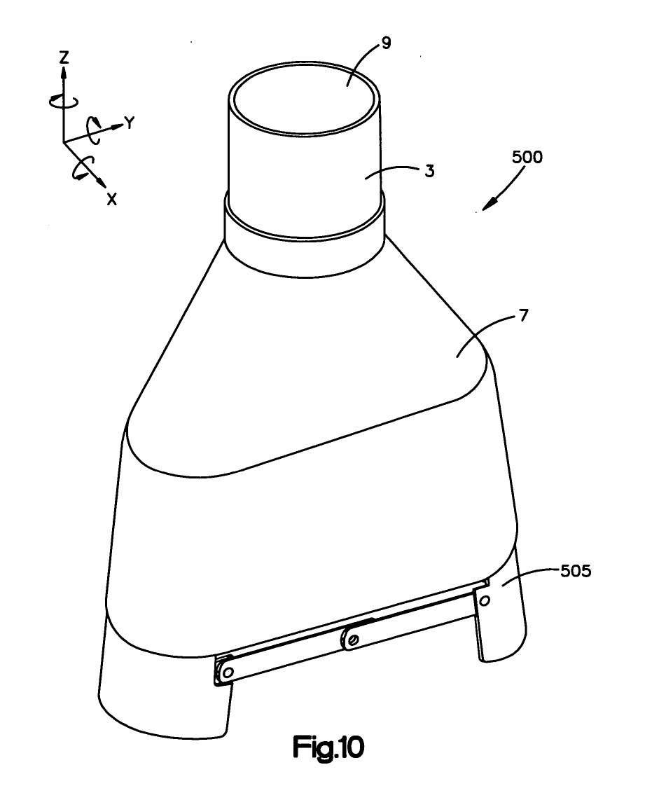

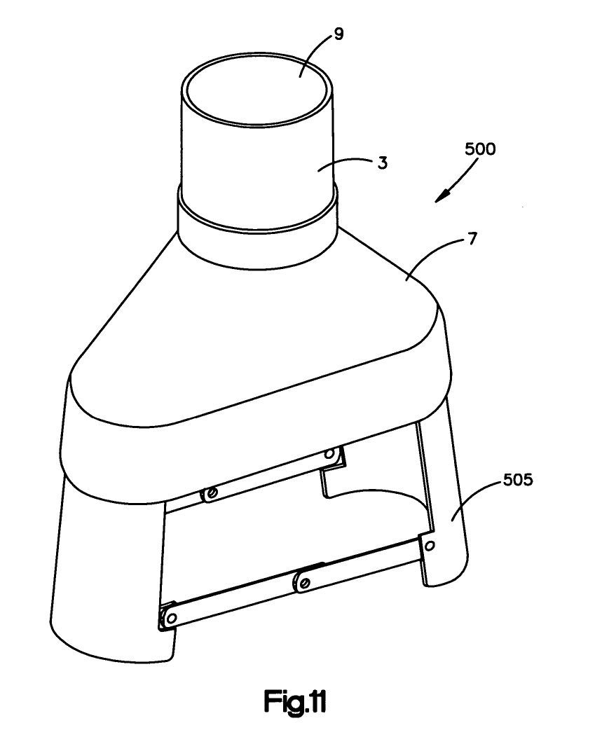

The expandable access device 500 may move between an unexpanded position, as best shown in FIG. 9, and an expanded position, as best shown in FIGS. 10 and 11. The mobility member 7 may stretch as the expandable portion 505 expands. The mobility member 7 may cover a majority of the expandable portion 505, as shown in FIG. 10, or a smaller portion of the expandable portion 505, as shown in FIG. 11. The size of the mobility member 7 may depend on the need to limit soft tissue intrusion into the working area 9a.

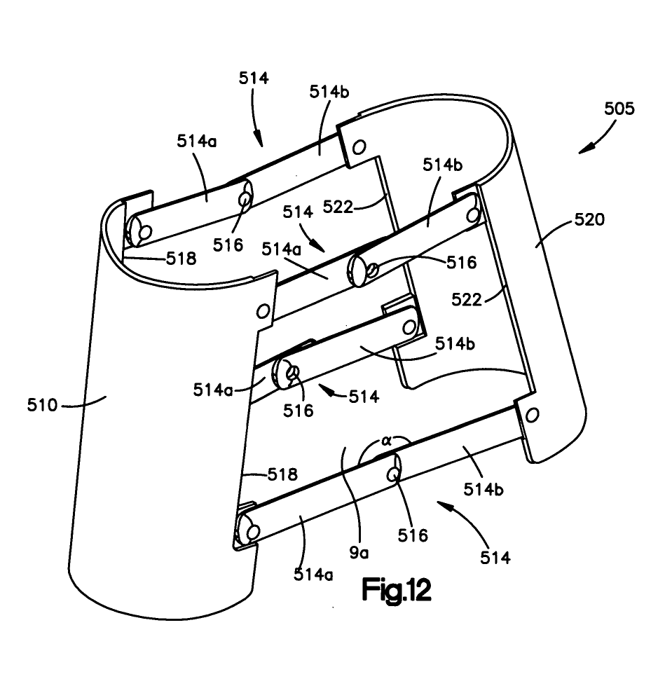

As shown in FIG. 12, the expandable portion 505 may include a first section 510 and a second section 520 wherein the first and second sections 510, 520 of the expandable portion 505 may be interconnected by one or more hinge members. The hinge members may be sized and configured so that the expandable portion deploys and/or collapses under the load of the mobility member 7. More specifically, the expandable portion 505 may include a first section 510 and a second section 520, wherein the first and second sections 510, 520 may be attached by two or more hinges 514. FIG. 12 shows an embodiment with four hinges 514. The hinges 514 may have a first hinge portion 514a and a second hinge portion 514b which may be pivotally connected to each other at pivot connection 516 and pivotally connected to first section 510 and second section 520, respectively. One or more hinges 514 may be selectively locked by, for example, opening the expandable portion 505 until the first and second hinge portions 514a, 514b may be at an angle α of about 180 degrees or less with respect to each other. It should be noted, however, that other means for selectively locking one or more hinges 514 are envisioned. The hinges 514 may be locked and unlocked and, the expandable portion 505 may be expanded and unexpanded, by a device (not shown) which may be inserted through the passage 9 and in between the first and second sections 510, 520.

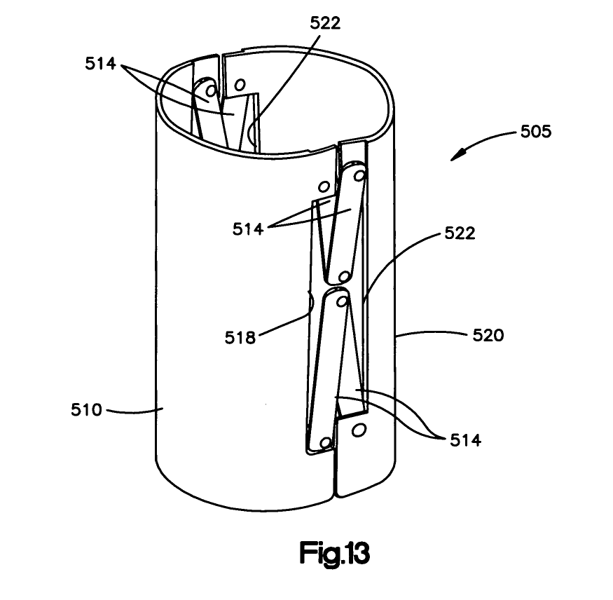

In the unexpanded position shown in FIG. 13, the first hinge portions 514a, 514b may be positioned in between first and second section 510, 520 within recesses 518 formed in the first section 510 and recesses 522 formed in the second section 520. In this way, the expandable portion 505 may be tubular in shape in the unexpanded position, and the first and second sections 510, 520 may be substantially flush with respect to each other. Further, in the unexpanded position, the expandable portion 505 may be sized and configured to fit within a tube (e.g., a cannula) which may have a dimension or diameter of, for example, at least about 30 mm. The tube may be used to insert the expandable access device 500 into the body and/or remove the device 500 from the body similar to the method described above for inserting/removing device 400.

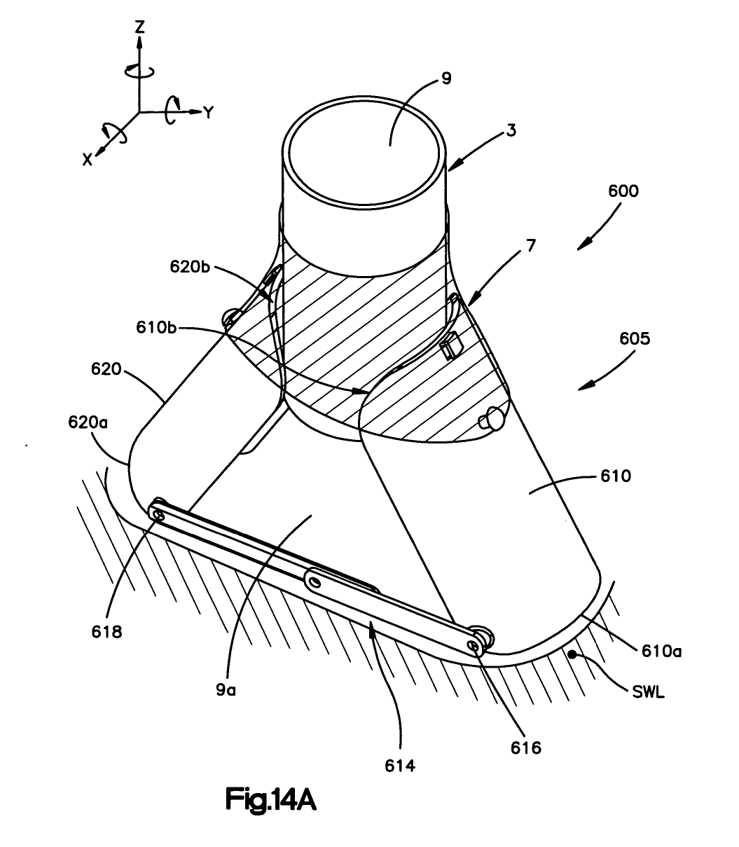

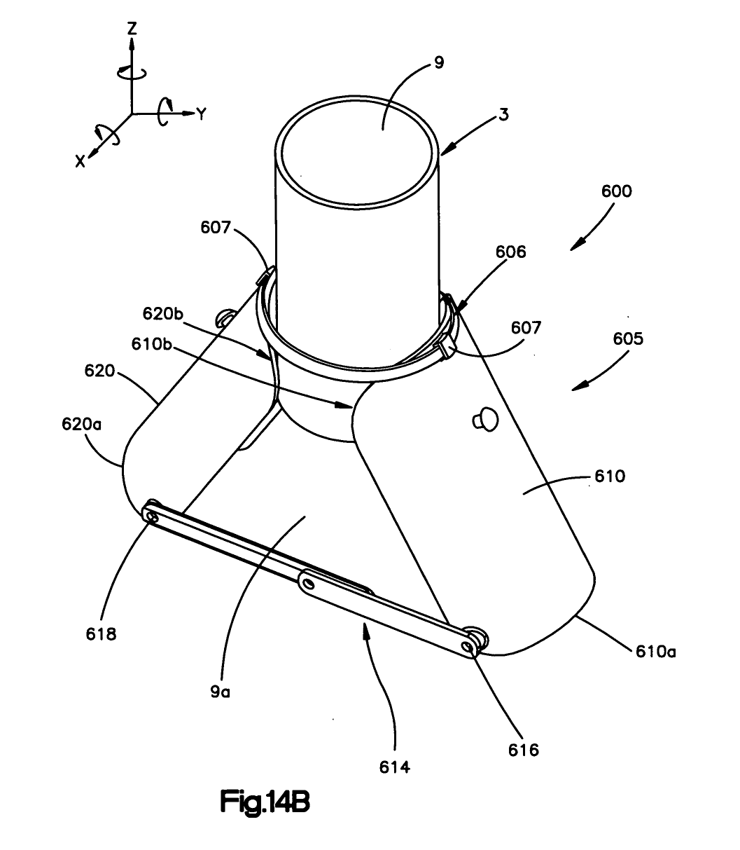

FIG. 14A illustrates an alternative expandable access device 600. As shown in FIG. 14A, the expandable device 600 may include an access portion 3, an expandable portion 605, and a mobility member 7, which may interconnect the access portion 3 to the expandable portion 605 so that the access portion 3 may move with respect to the expandable portion 605 (e.g., the access portion 3 may translate along, angulate about and/or rotate about the x, y and/or z axis). The expandable access device 600 may also include a passage 9 extending therethrough.

The expandable portion 605 may include a first section 610 and a second section 620 wherein the first and second sections 610, 620 of the expandable portion 605 may be interconnected by one or more hinge members. For example, the first and second sections 610, 620 may be attached by a hinge 614 proximate the distal ends 610a, 620a of the first and second sections 610, 620, respectively. The hinge 614 may be attached to first section 610 at pivot 616 and the second section 620 at pivot 618. The pivots 616, 618 may provide points of rotation for the first and second sections 610, 620. The proximal ends 610b, 620b of the first and second sections 610, 620, respectively, may be positioned adjacent the access portion 3 so that the first and second sections 610, 620 rest against the access portion 3. In such a configuration the expandable portion 605 may be semi-constrained to the access portion 3.

In an alternative embodiment, as shown in FIG. 14B, the access portion 3 and the expandable portion 605 may be attached by a tether 606. In one embodiment, the tether 606 may be an elastomeric band. Similar to the mobility member 7, the tether 606 may allow the access portion 3 to translate along, angulate about and/or rotate about the x, y and/or z axis. Moreover, the tether 606 may semi-constrain the expandable portion 605 to the access portion 3. The tether 606 may be made from any material known in the art including, but not limited to, plastic, rubber, metal, a composite, etc. The tether 606 may be positioned through holding members 607 which may be connected to the first and second section 610, 620. The tether 606 may be elastic such that the tether 606 may exert a force on the first and section sections 610, 620 and, thereby, may keep the proximal ends 610b, 620b of the first and second section 610, 620, respectively, in contact with the access portion 3. It will be appreciated by those skilled in the art that other means of semi-constraining the expandable portion 605 to the access portion 3 are envisioned.

Using the embodiments of FIGS. 14A and 14B, translation of the access portion 3 along the y-axis may result in the first and second sections 610, 620 rotating about pivots 616 and 618, respectively (the first and second sections 610, 620 may rock back and forth). The pivots 616, 618 may remain fixed at surgical work level SWL, thereby maintaining the working area 9a. Moreover, the proximal ends 610b, 620b of the first and second sections 610, 620, respectively, may remain in contact with the access portion 3 and may slide along the access portion 3.

To perform a surgical procedure, a surgeon makes an incision into a patient's body, which may be, for example, between about 10 mm and about 50 mm, preferably between about 20 mm and about 40 mm. In one embodiment, the incision may be sequentially dilated by inserting a series of dilators into the incision. A cannula may thereafter be positioned over the largest dilator and the dilators may be removed, leaving the cannula in the incision. Thereafter, the expandable access device 1, 100, 200a, 300, 400, 500, 600 may be positioned over or into the cannula and may be inserted through the incision down to a surgical site. Once the expandable device 1, 100, 200a, 300, 400, 500, 600 is in position, the cannula may be removed, thereby leaving the device 1, 100, 200a, 300, 400, 500, 600 in the body.

Alternatively, when the incision has been dilated enough to insert the device 1, 100, 200a, 300, 400, 500, 600 into the body, the largest dilator may remain in the incision while the other dilators may be removed. Thereafter, the device 1, 100, 200a, 300, 400, 500, 600 may be inserted over or through the largest dilator. In such an embodiment, a cannula may be unnecessary. The largest dilator may be removed after the device 1, 100, 200a, 300, 400, 500, 600 is positioned in the body. In another embodiment, an obturator may be positioned inside the passage 9 of the device 1, 100, 200a, 300, 400, 500, 600. The obturator and device 1, 100, 200a, 300, 400, 500, 600 may be inserted into the body as a unit. This step may be performed with or without sequential dilation of the incision. After insertion of the device 1, 100, 200a, 300, 400, 500, 600 into the body, the obturator may be removed, leaving the device 1, 100, 200a, 300, 400, 500, 600 in the body. In another embodiment, the device 1, 100, 200a, 300, 400, 500, 600 may be positioned in a tube (e.g., cannula) and inserted in the body as a single unit.

The expandable portion 5, 105, 205, 205a, 305, 405, 505, 605 may be positioned adjacent the surgical site. Thereafter, the expandable portion 5, 105, 205, 205a, 305, 405, 505, 605 may be moved to an expanded condition, thereby stretching the mobility member 7. A surgical procedure may then be performed through the passage 9 and the access portion 3 may be translated along, angulated about and/or rotated about the x, y and/or z-axis. After the procedure has been completed, the expandable portion 5, 105, 205, 205a, 305, 405, 505, 605 may be moved to the unexpanded position and removed from the body.

While the foregoing description and drawings represent the preferred embodiments of the present invention, it will be understood that various additions, modifications and substitutions may be made therein without departing from the spirit and scope of the present invention as defined in the accompanying claims. In particular, it will be clear to those skilled in the art that the present invention may be embodied in other specific forms, structures, arrangements, proportions, and with other elements, materials, and components, without departing from the spirit or essential characteristics thereof. One skilled in the art will appreciate that the invention may be used with many modifications of structure, arrangement, proportions, materials, and components and otherwise, used in the practice of the invention, which are particularly adapted to specific environments and operative requirements without departing from the principles of the present invention. The presently disclosed embodiments are therefore to be considered in all respects as illustrative and not restrictive, the scope of the invention being indicated by the appended claims, and not limited to the foregoing description.

What is claimed is:

1. An expandable access device for providing access from the exterior to the interior of a living body comprising:
an access portion having a proximal end, a distal end and a substantially hollow passageway extending longitudinally from the proximal end to the distal end;
an expandable portion having a proximal end, a distal end and a substantially hollow passageway extending longitudinally from the proximal end to the distal end, the distal end capable of being positioned proximate a surgical site, the expandable portion has an unexpanded position and an expanded position, wherein the distal end of the expandable portion in the expanded position has a larger cross-sectional area than in the unexpanded position; and
a mobility member operably connecting the access portion and the expandable portion, wherein the mobility member enables at least one of translating and rotating of the access portion relative to the expandable portion;
wherein the passageway of the access portion communicates with the passageway of the expandable portion.

2. The expandable access device of claim 1, wherein at least a portion of the mobility member expands as the expandable portion moves to the expanded position and contracts as the expandable portion moves to the unexpanded position.

3. The expandable access device of claim 1, wherein the access portion has a predetermined, fixed cross-sectional area.

4. The expandable access device of claim 1, wherein the access portion is expandable.

5. The expandable access device of claim 4, wherein the access portion is deformable.

6. The expandable access device of claim 1, wherein the mobility member is made from at least one of the materials selected from the group consisting of plastic, rubber, elastomer, polymer, metal and a composite.

7. The expandable access device of claim 6, wherein the material is selected from at least one of the group consisting of silicone rubber and urethane,

8. The expandable access device of claim 1, wherein the mobility member enables angulation of the access portion relative to the expandable portion.

9. The expandable access device of claim 1, wherein the ends of the access portion and the expandable portion have a perimeter, wherein the mobility member is a thin sheet of elastomeric material surrounding the perimeter of at least one of the ends of the expandable portion and the access portion.

10. The expandable access device of claim 1, wherein the expandable portion is moved between the expanded and unexpanded position in increments.

11. The expandable access device of claim 1, wherein the expandable portion has a first and second member, wherein each member has a receiving portion and a flap such that the flap of the first member is receivable in the receiving portion of the second member and the flap of the second member is receivable in the receiving portion of the first member.

12. The expandable access device of claim 11, wherein the flap of at least one of the first and second member comprises a plurality of teeth.

13. The expandable access device of claim 1, wherein the expandable portion has at least one hinge.

14. The expandable access device of claim 1, wherein the expandable portion has a first section and a second section, the first and second sections interconnected by one or more hinge members.

15. The expandable access device of claim 1, wherein the mobility member has a proximal end and a distal end, wherein the proximal end of the mobility member is connected at a location on the access portion and the distal end of the mobility member is connected proximate the proximal end of the expandable portion.

16. The expandable access device of claim 1, wherein the mobility member has a proximal end and a distal end, wherein the proximal end of the mobility member is connected at a location on the access portion and the distal end of the mobility member is connected proximate the distal end of the expandable portion.

17. The expandable access device of claim 1, wherein the expandable portion comprises a plurality of blades.

18. The expandable access device of claim 17, wherein at least two blades have a curved shape.

19. The expandable access device of claim 18, wherein the curved blades in the unexpanded position substantially surround and define the passageway through the expandable portion.

20. The expandable access device of claim 19, wherein the blades have edges such that in the unexpanded position, the edges of adjacent blades are proximate each other and in the expanded position, at least distal edges of adjacent blades are a distance from each other, thereby creating openings between the blades.

21. The expandable access device of claim 20, wherein the mobility member extends between the edges of adjacent blades such that the mobility member surrounds and defines at least a portion of passageway of the expandable portion.

22. The expandable access device of claim 17, wherein adjacent blades are connected at a pivot point.

23. The expandable access device of claim 1, wherein the access portion has multiple degrees of freedom of movement relative to the expandable portion.

24. The expandable access device of claim 1, wherein the access portion has a first dimension perpendicular to its passageway and the expandable portion a second dimension at its distal end perpendicular to its passageway, the first dimension being substantially the same as the second dimension when the expandable portion is in the unexpanded position.

25. The expandable access device of claim 24, wherein the first dimension is different than the second dimension when the expandable portion is in the expanded position.

26. An expandable access device for providing access from the exterior to the interior of a living body comprising:
an access portion having a proximal end and a distal end;
an expandable portion having a first end, a second end and at least one holding member operably associated with at least a portion of the expandable member, the second end capable of being positioned proximate a surgical site, the expandable portion has an unexpanded position and an expanded position, wherein the second end of the expandable portion in the expanded position has a larger cross-sectional area than in the unexpanded position; and
a tether operably connected to the at least one holding member, wherein the tether and at least one holding member enables at least one of translation and rotation of the access portion 11 relative to the expandable portion.

27. The expandable access device of claim 26, wherein the expandable portion comprises first and second sections wherein each section has a holding member proximate the first end of the expandable portion and wherein the tether is connected to the holding members of the first and second sections.

28. The expandable access device of claim 26, wherein the tether is made from at least one of the materials selected from the group consisting of plastic, rubber, polymer, elastomer, metal and a composite.

29. The expandable access device of claim 26, wherein the tether is a elastomeric band.

30. A method of providing an access portal from exterior of the body to an interior location in the body to be accessed by medical instruments, comprising:
providing an expandable access device comprising
an access portion having a first longitudinal axis;
an expandable portion having a second longitudinal axis, a proximal end, a distal end, an unexpanded position and an expanded position, wherein the distal end of the expandable portion in the expanded position has a larger cross-sectional area than in the unexpanded position; and
a connecting member operably connecting the access portion and the expandable portion;
inserting at least a portion of the expandable portion of the access device into the body while the expandable portion is in the unexpanded position;
manipulating the distal end of the expandable portion to the interior location to be accessed;
forming a passageway extending between the access portion and the expandable portion;
inserting at least one medical instrument into the passageway to the interior location; and
performing at least one of rotating about one of the first and second longitudinal axes and translating the first and second longitudinal axes relative to each other.

31. The method of claim 30 further comprising angulating the access portion relative to the expandable portion.

32. The method of claim 31, the method further comprising simultaneously performing at least two of translating, rotating and angulating the access portion relative to the expandable portion.

33. The method of claim 30 further comprising expanding the access portion.

34. The method of claim 30 further comprising expanding the expandable portion.

35. The method of claim 30 further comprising moving the access portion with at least one of a finger and the one or more surgical or diagnostic instruments to palpate patient anatomy.

36. The method of claim 30 wherein the step of moving the expandable portion comprises moving the expandable portion in increments between the expanded and unexpanded position.

37. The method of claim 30 wherein the passageway defines an access space when the expandable portion is in the expanded position, the method further comprising preventing unresected tissue from entering at least a portion of the access space.

38. The method of claim 30, the method further comprising polyaxially angulation the access portion relative to the expandable portion.
